(19) United States
(12) Patent Application Publication
(10) Pub. No.: US 2005/0043796 A1
(43) Pub. Date: Feb. 24, 2005

(54) Spinal disc nucleus implant

(76) Inventors: Richard L. Grant, Cincinnati, OH (US); Joseph E. Young, Loveland, OH (US); Steven L. Henderson, Loveland, OH (US)

Correspondence Address: JERROLD J. LITZINGER, 2134 MADISON ROAD, CINCINNATI, OH, 45208, US

(21) Appl. No.: 10/883,223

(22) Filed: Jul. 1, 2004

Related U.S. Application Data
(60) Provisional application No. 60/484,091, filed on Jul. 1, 2003.

(51) Int. Cl. A61F 2/44

(52) U.S. Cl. 623/17.11; 623/17.16; 606/86

(57) ABSTRACT

A device and method for repairing spinal discs in which the nucleus has been damaged. A central section of the nucleus is cored out using a device which ablates the tissue using RF energy. A nucleus implant comprising a shape memory material is placed in the central section of the disc through a cannula, which implant is activated by body heat to expand and fill the central section, such that the implant emulates the functions and strength of the disc's natural nucleus.

### CROSS REFERENCE TO RELATED APPLICATIONS

This application claims benefit from U.S. Provisional Application Ser. No. 60/484,091, filed Jul. 1, 2004, which application is incorporated herein by reference.

### BACKGROUND OF THE INVENTION

1. Field of the Invention

The present invention relates generally to spinal implants, and, in particular, to devices and methods for repairing discs in which the nucleus has been damaged.

2. Description of the Prior Art

Due to strain or degenerative disease, the nucleus of a spinal disc often herniates or erupts through the annulus of the disc. This action weakens the disc, as well as creating tremendous pain to the patient as the hernia “bulge” creates pressure on the spinal nerve bundles.

There are currently two common practices that are employed by surgeons to correct this situation:
1) remove the hernial bulge by nibbling or coring away the tissue which is pressing on the nerve bundles. While this procedure is minimally invasive, the disc is weakened and compressed due to loss of nucleus tissue, and it also leaves a path for future disc herniation problems;
2) fusing the spine by the application of spine screws and securing rods to the surrounding vertebrae to extrapolate the damaged disc. While this procedure provides rigid support for the spine at the damaged disc area, it is very invasive, and limits the patient's range of motion. In addition, it is limited to repairs on the lumbar vertebrae.

There have been many attempts in recent years to replace the damaged nucleus of a spinal disc. U.S. Patent Application Publication No. US2002/0183848A is directed to a spinal disc nucleus having a hydrogel core surrounded by a constraining jacket. The hydrogel core is configured to expand from a dehydrated state to a hydrated state. The dehydrated core is implanted through an annulus opening and then hydrated to fill the constraining jacket.

U.S. Patent Application Publication No. 2002/004800A is directed to a prosthetic pulposus for replacing the natural nucleus pulposus of an intervertebral disc. The prosthetic device comprises a partially collapsed sealed envelope formed from material permeable to extracellular body fluid. The envelope is implanted in the hollowed out interior of the disc and is allowed to absorb fluid, whereby expanding the envelope.

U.S. Patent Application Publication No. 2003/0199984A is directed to nucleus pulposus implants that are resistant to migration from an intervertebral disc space. The implants include a load bearing elastic body surrounded in the disc space by an anchoring, preferably resorbable, outer shell.

U.S. Patent Application Publication No. 2004/0030392A is directed to a system for minimally invasive disc augmentation having an annulus augmentation component and a nucleus augmentation component. The nucleus augmentation component restores disc height and/or replaces missing nucleus pulposus. This may be supported by inserting a flexible biocompatible material into the disc space.

U.S. Patent Application Publication No. 2004/0054413A is directed to a spinal implant for replacing the natural nucleus of the disc made from a polymer such as hydrogel having a radiopaque material located within the polymer. The material may be in the form of a powder dispersed throughout the polymer or dispersed in layers within the polymer.

U.S. Patent Application Publication No. 2004/0091540A is directed to a minimally invasive method for restoring a damaged or degenerated intervertebral disc at an early stage. An injectable in situ setting formulation in the nucleus pulposus combines with nucleus matter and host cells to gel within the annulus fibrosis of the disc for increasing the thickness and volume of the damaged disc.

U.S. Patent Application Publication No. 2004/0117019A is directed to a method of implanting a prosthetic spinal disc nucleus by providing an instrument having a dilator for dilating an opening in the disc annulus and passing the prosthetic disc nucleus into the disc through the dilator in one configuration and allowing the disc to assume another configuration when implanted within the annulus.

In the replacement of the nucleus of a spinal disc, it is imperative that the implant be precisely centered within the nucleus to accept the natural movement and forces that are generated by normal patient activity, and must be retained in this location such that it is centered on the line of force to prevent the implant from placing undue forces on the annulus.

### SUMMARY OF THE INVENTION

It is therefore an object of the present invention to provide a method for accurately removing the nucleus of a spinal disc which is minimally invasive.

It is a further object of the present invention to provide a spinal disc nucleus implant which emulates the functions and strengths of the spinal disc's natural nucleus.

It is a still further object of the present invention to provide a device for implanting the replacement nucleus into the proper location within the cored-out disc easily and safely.

These and other objects of the present invention will be more readily apparent from the description and drawings which follow.

### BRIEF DESCRIPTION OF THE DRAWINGS

FIG. 1 is a top view of the typical spinal disc showing a central core within the nucleus;

FIG. 2 is a perspective view of a device for creating a core within the nucleus of a spinal disc;

FIG. 3 is a perspective view of the device of FIG. 2 with the electrode inflated;

FIG. 4 is a perspective view of another embodiment similar to the device of FIG. 2;

FIG. 5 is a perspective view of the device of FIG. 4 with the electrode expanded;

FIG. 6 is a top view of another embodiment of a device according to the present invention;

FIG. 7 is a top view of another embodiment of a device similar to the device of FIG. 6;

FIG. 8 is a top view of another embodiment of a device similar to the device of FIG. 6;

FIG. 9 is a plan view of a nucleus implant in its pre-insertion state;

FIG. 10 is a plan view of the implant of FIG. 9 in its implanted state;

FIG. 11 is a perspective view of another embodiment of the present invention in its pre-insertion state;

FIG. 12 is a perspective view of the implant of FIG. 11 in its implanted state;

FIG. 13 is a perspective view of another embodiment of the present invention in its pre-insertion state;

FIG. 14 is a perspective view of the insertion of the implant of FIG. 13;

FIG. 15 is a plan view of the implant of FIG. 13 installed between two vertebrae;

FIG. 16 is a perspective view of another embodiment of the present invention shown in its implanted state;

FIG. 17 is a sectional view of the device of FIG. 16 shown in its installed position;

FIG. 18 is a cross-sectional view taken along line 18-18 in FIG. 17;

FIG. 19 is a perspective view of another embodiment of the present invention shown in its uninstalled state;

FIG. 20 is a perspective view of the device of FIG. 19 shown in its implanted state;

FIG. 21 is a perspective view of FIG. 20 showing its cross section;

FIG. 22 is a perspective view of another embodiment of the present invention shown in its uninstalled state;

FIG. 23 is a perspective view of the device of FIG. 22 shown in its implanted state;

FIG. 24 is a perspective view of FIG. 23 showing its cross section;

FIG. 25 is a perspective view of another embodiment of the present invention shown in its uninstalled state;

FIG. 26 is a perspective view of the device of FIG. 25 shown in its implanted state;

FIG. 27 is a perspective view of FIG. 26 showing its cross section;

FIG. 28 is a perspective view of a shape memory material core suitable for use in the present invention;

FIG. 29 is a plan view of basket suitable for use in the present invention;

FIG. 30 is a fragmentary view of an alternate end for use in the device of FIG. 29;

FIG. 31 is top view, partly in cross-section, of a device for use in performing a procedure according to the present invention;

FIG. 32 is a top view, partly in cross section, of the device shown in FIG. 31 used for installing a device of the present invention;

FIG. 33 is a plan view of another embodiment of the present invention; and

FIG. 34 is a plan view of the device of FIG. 33 shown in its implanted state.

### DETAILED DESCRIPTION OF THE EMBODIMENTS

Referring now to FIG. 1, there is shown a representative spinal disc, generally indicated at 10. Disc 10 contains a nucleus 12 and an annulus 14. Within nucleus 12 is a centrally located cored out section 16. Section 16 is created to precisely locate an artificial nucleus implant which would accept a standard shape. Section 16, which houses the implant, should ideally be centered in the nucleus to accept the natural movement and forces that are generated by normal patient activity.

To define a standard core section 16 shape, an instrument must be developed which can consistently core out this area within nucleus 12. The instrument should be small in size such that the procedure can be performed as minimally invasive. It is important that the nucleus implant remains centered on the line of force, preventing it from placing undue forces on annulus 14. One method for accomplishing these objectives is to leave annulus 14 in place and some of the tissue of nucleus 12 but to locate an exact cavity 16 into which the implant is fitted.

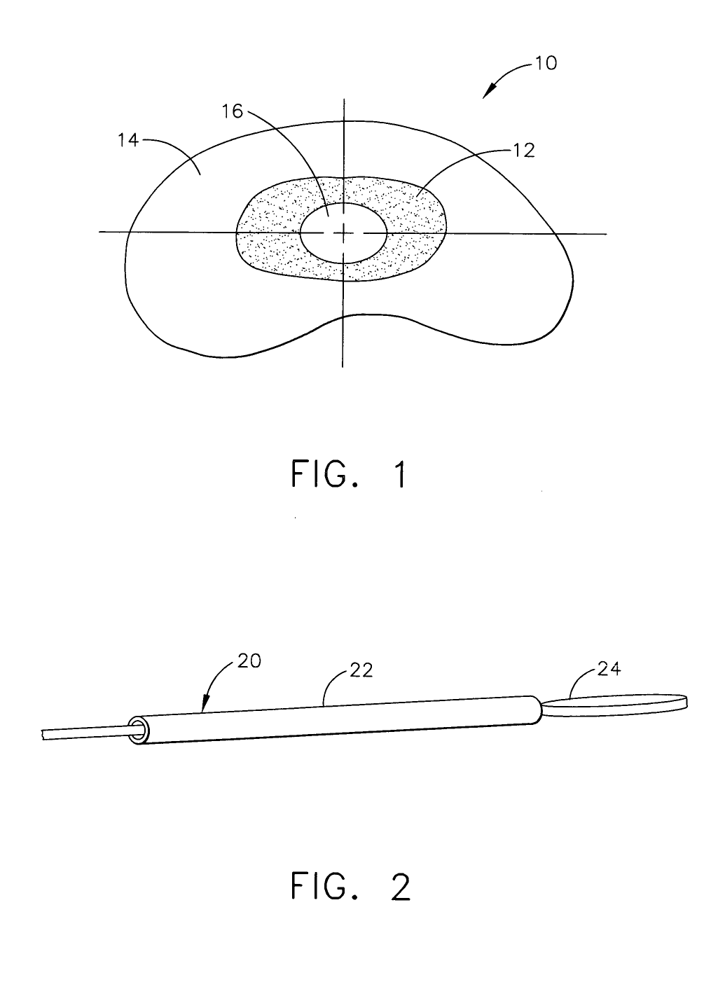

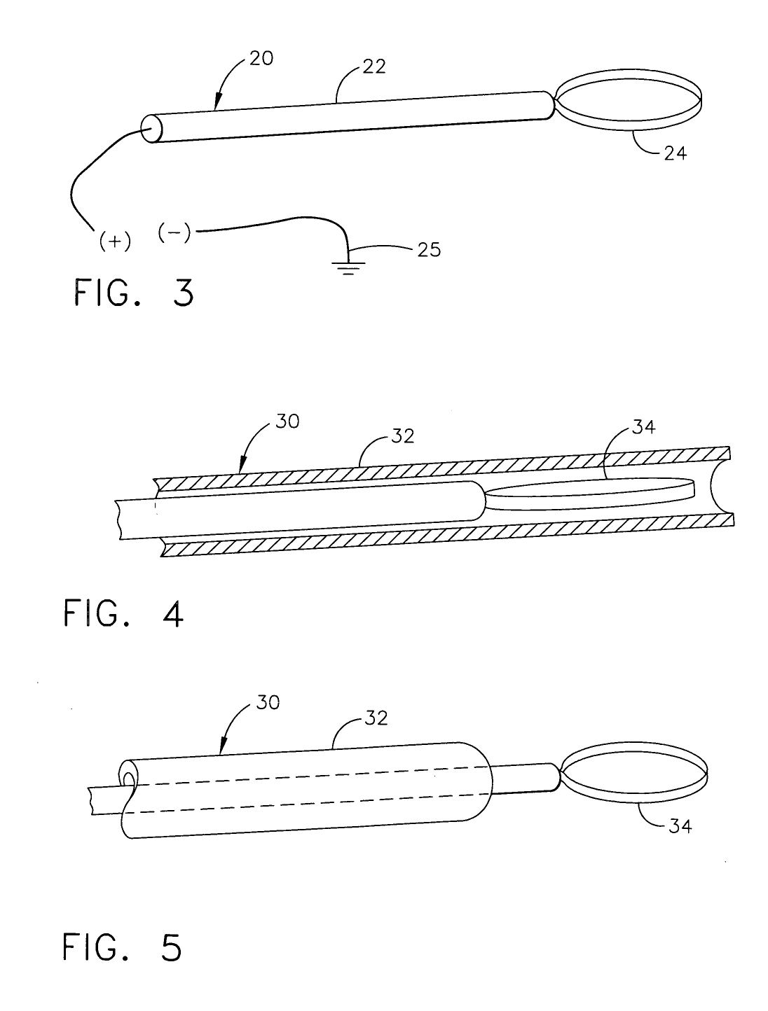

One method for removing the tissue within nucleus 12 is by ablation. RF energy may be used to power an electrode that is the size and shape of the cavity desired. FIGS. 2 and 3 show an instrument 20 suitable for use in forming core section 16 within nucleus 12. Instrument 20 consists of a probe 22 having a collapsed electrode 24 at its tip. Instrument 20 can be inserted endoscopically into the disc until electrode 24 is at its desired location. Electrode 24, which is a balloon in this embodiment, is slowly inflated using a gas or liquid, ablating the tissue as it grows. Electrode 24 acts as the positive electrode while the ground electrode is connected to the patient. Irrigation or evacuation could be used to remove gases generated during ablation.

FIGS. 4 and 5 show another embodiment for an ablating instrument for use in the present invention. Instrument 30 contains a hollow shaft 32 within which a collapsed electrode 34 is stored. When the tip of instrument 30 reaches the desired destination, electrode 34, which may consist of spring band, is expanded, ablating the nucleus tissue.

The aforementioned techniques and instruments would need to be centered in the disc. This may be accomplished by measuring the center line or by interoperative imaging. Additionally, hooked or straight probes may be employed to find the extremes of the disc and then measured to the center. Guidance and imaging will be critical to centrically locate core section 16 within the disc to insure that the nucleus implant performs optimally.

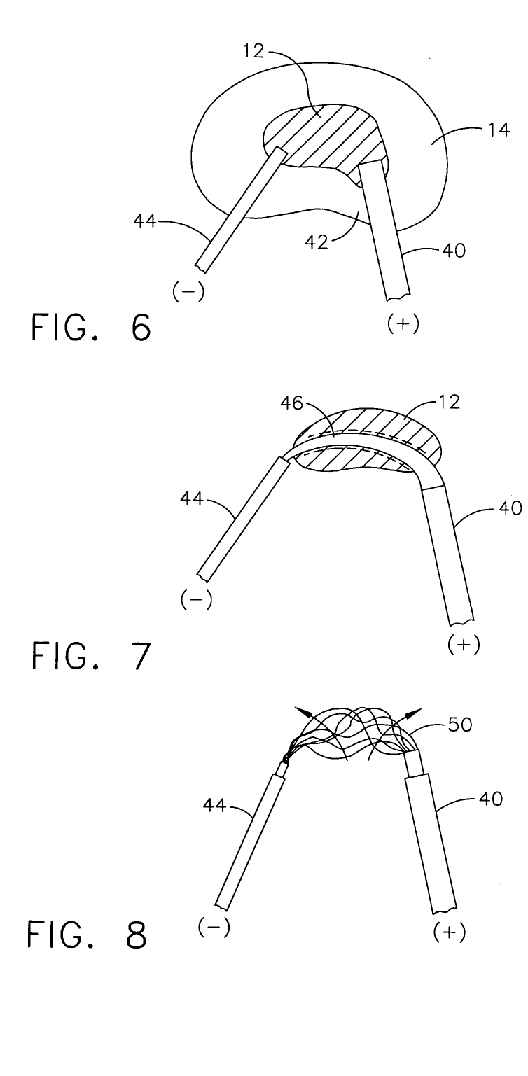

Another technique for creating a cored out section within the nucleus is shown in FIGS. 6, 7, and 8. Referring now to FIG. 6, there is shown a first cannula 40 inserted through a herniated area 42 within annulus 14 into nucleus 12. A second smaller cannula 44 may be inserted through a healthy section of annulus 14 into nucleus 12. Cannulae 40 and 44 may act as positive and negative electrodes for an RF device, and cannula 44 helps to find the proper location.

An RF device 46 is deployed out of cannula 40 and the distal end inserted or retracted into cannula 44. Device 46 may be a balloon-like catheter with a conductive coating or flexible wires that could be energized to ablate the nucleus tissue. Cannulae 40, 44 may also have a vacuum port to remove gases and/or tissue. Device 46 may also consist of a series of wires 50, as shown in FIG. 8, or a woven basket, that could be energized and rotated to core out tissue. As wires 50 are energized with RF energy, the woven basket would be slowly rotated.

The major goal of the aforementioned devices and procedures is to core out a standard shape centrically within the disc nucleus and insert an implant that will support and give full motion, emulating the spinal disc's natural nucleus.

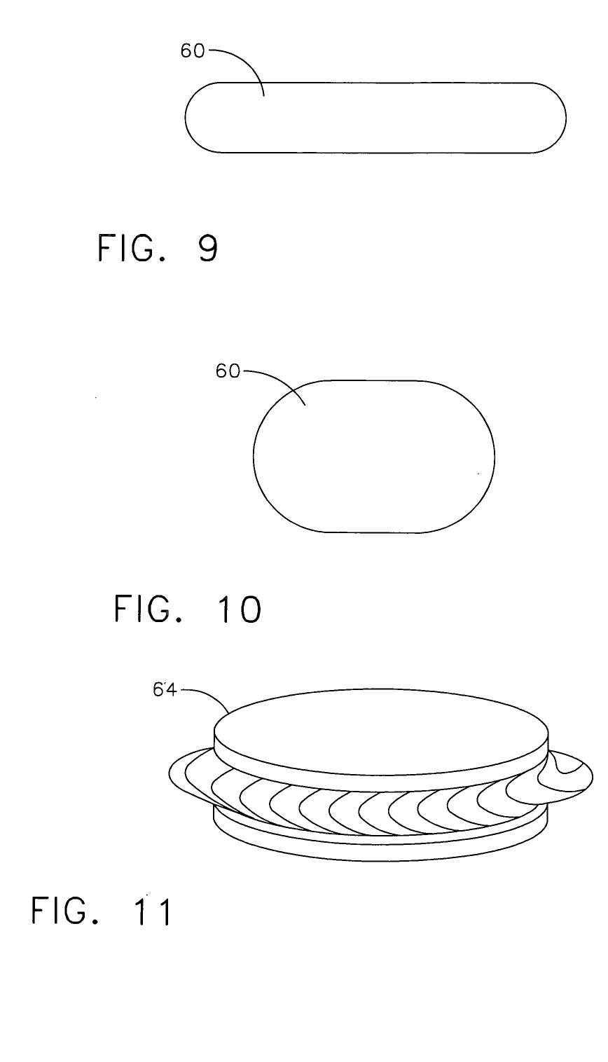

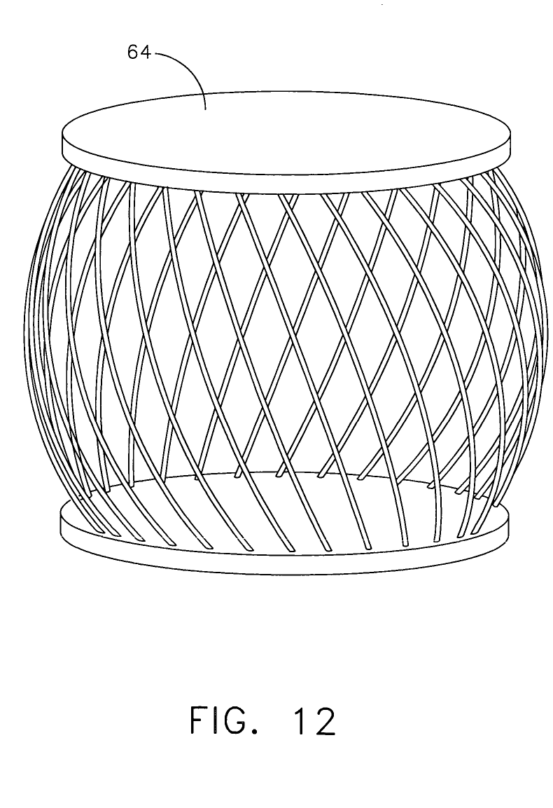

The second element of the present invention is the implant. The size and shape of the implant is determined by the core shape formed with the aforementioned instruments. FIGS. 9 and 10 show a nucleus implant constructed according to the present invention. FIG. 9 shows an implant 60 in its pre-insertion state. Implant 60 is composed of a material having shape memory properties which will be activated by external means such as thermal mans, light means, RF means, or chemical means. Acceptable shape memory materials are available from the Polymer Technolgy Group of Berkeley, Calif. and mnemoScience GmbH of Aachen, Germany. Before the material is activated, it will be small in size for ease of insertion into the created core. Upon activation, implant 60 will change shape to fit within the cored out area of the disc, as seen in FIG. 10. As can be seen in the FIGS., before material 60 is activated, it will be small in size for ease of insertion into the created core 16 in disc 10, and then activated by swelling or expanding to populate core 16.

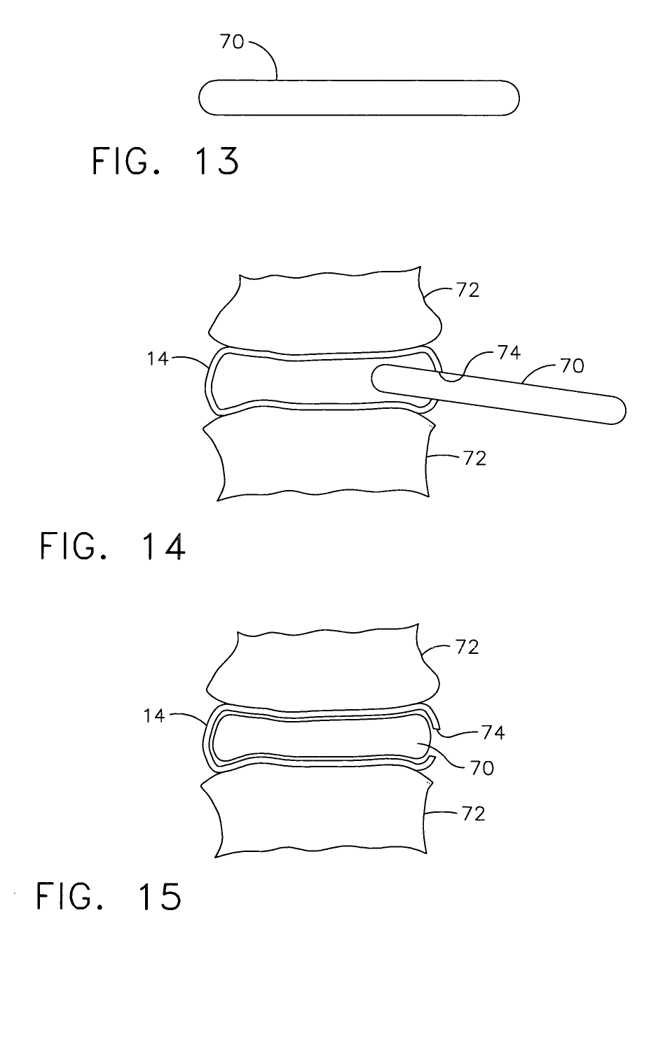

Another embodiment of an implant for use in the spinal disc is shown in FIGS. 11 and 12. Referring now to FIG. 11, there is shown a shell or stent-like basket 64 created using a metallic material which contains a shape memory material within the basket. After insertion into core 16 of disc 10, the shape memory material within basket 64 expands to fill core 16.

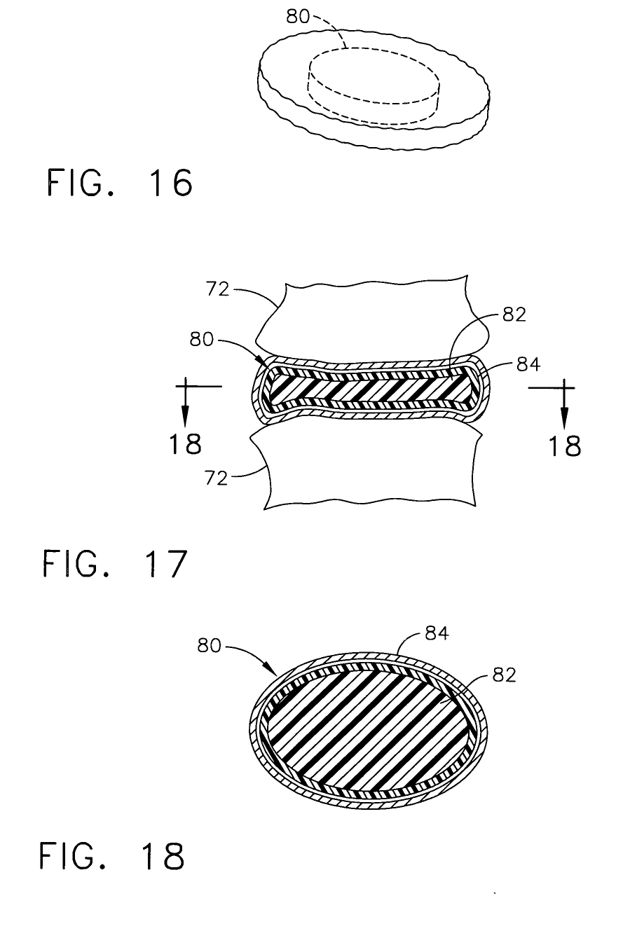

The use of an implant constructed from shape memory material within the spine is shown in FIGS. 13, 14, and 15. Implant 70 is shown in FIG. 13 in a shape, prior to insertion, at room temperature or below. In FIG. 14, implant 70 is inserted between two vertebrae 72, which are L4 and L5 in this embodiment. Implant 70 is inserted through a hole 74 in annulus 14 of the damaged disc. As implant 70 warms to body temperature, the shape memory polymer expands to fill the space cored out within the disc. After implant 70 expands, it will not be able to extrude through hole 74 in annulus 14.

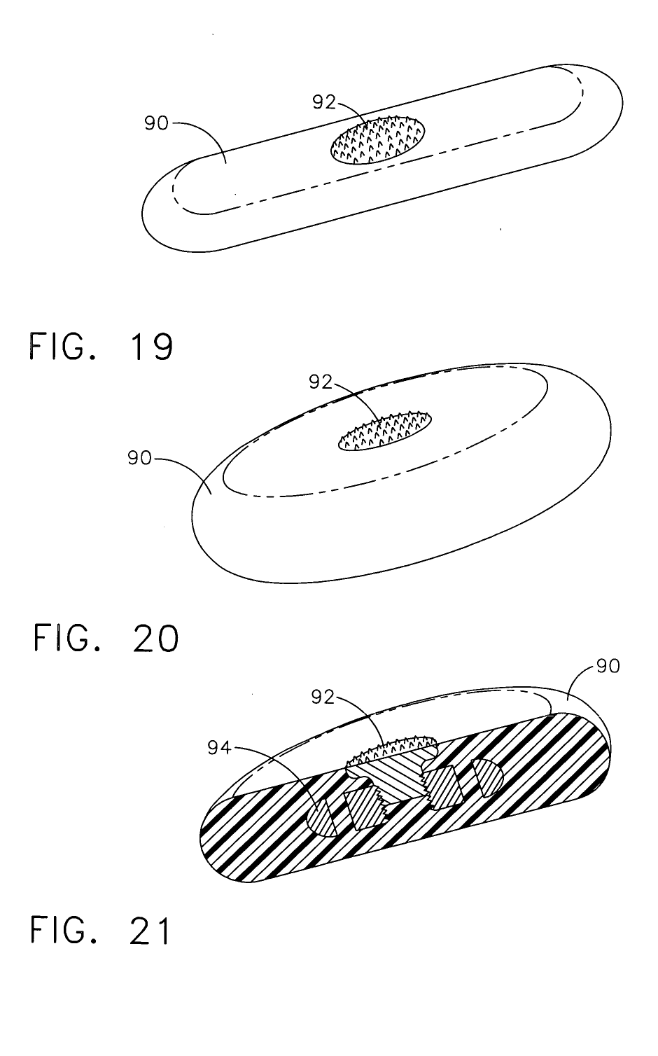

FIGS. 16, 17, and 18 show an implant 80 according to the present invention in position within a disc 10 between vertebrae. FIG. 16 shows implant 80 in its expanded shape at body temperature. FIG. 17 shows implant 80 located between vertebrae 72 surrounded by annulus 14. In this embodiment, shape memory material 82 is encapsulated within a shell 84.

When the implant is molded in its cold or inactivated state, stabilizing elements can be included such that when the material assumes its activated state, these elements assist in anchoring the implant in its proper place. Examples of these elements can be seen in FIGS. 19-27.

FIGS. 19, 20, and 21 show a nucleus disc implant 90 in which a stabilizing plate 92 is incorporated into the shape memory material. FIG. 19 shows implant 90 in its constrained state, while FIGS. 20 and 21 show implant 90 in its implanted state. Plate 92, which is attached to an anchor 94 molded within implant 90, contacts the lower surface of the vertebra above it to assist in holding implant 90 in its proper location.

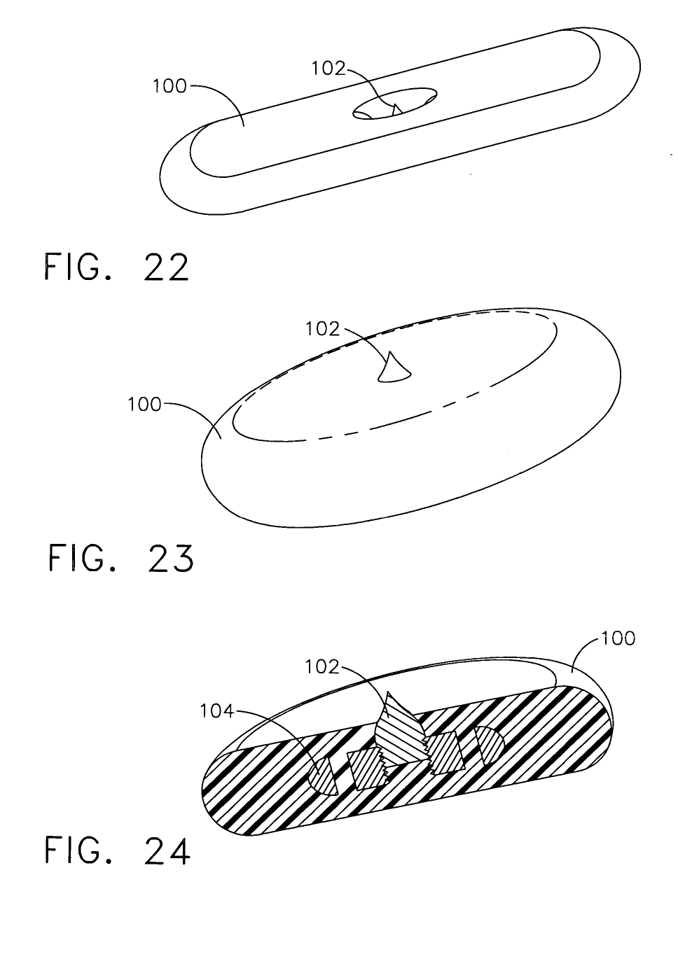

FIGS. 22, 23, and 24 show a disc implant 100 having a central prong 102 which serves to affix implant 100 in its proper location in its implanted state. Prong 102 which extends upwardly in its implanted state (FIG. 24), is attached to an anchor 104 molded within implant 100. Prong 102 extends upwardly to affix implant 100 in its proper position by contacting the vertebra directly above it.

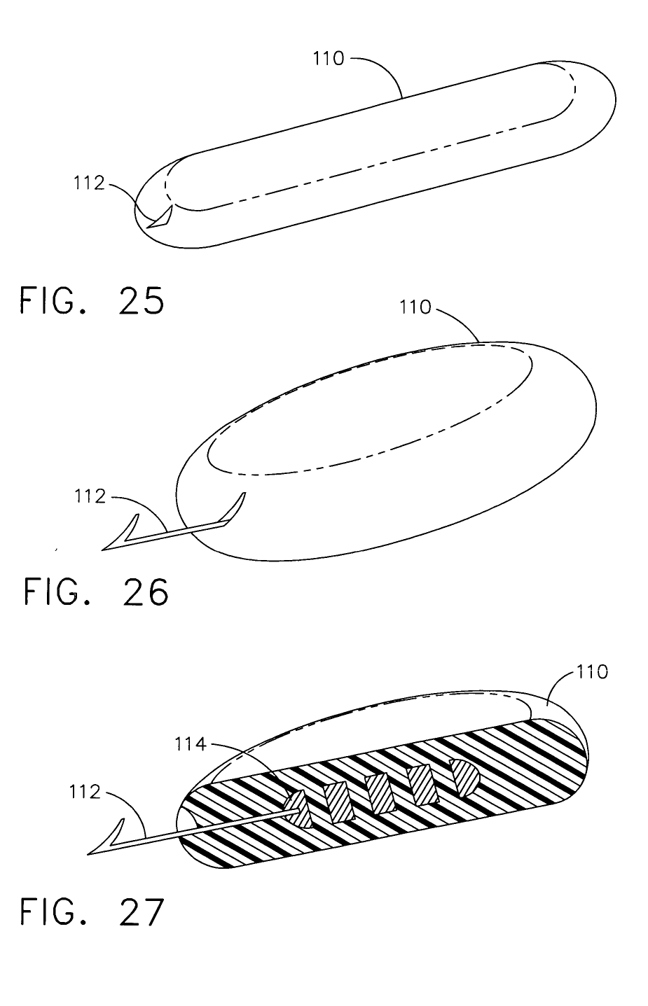

FIGS. 25, 26, and 27 show a disc implant 110 having a central prong 112 which extends laterally from the implant to affix implant 110 in its proper position. Prong 112 is attached to an anchor 114 which is molded into implant 110.

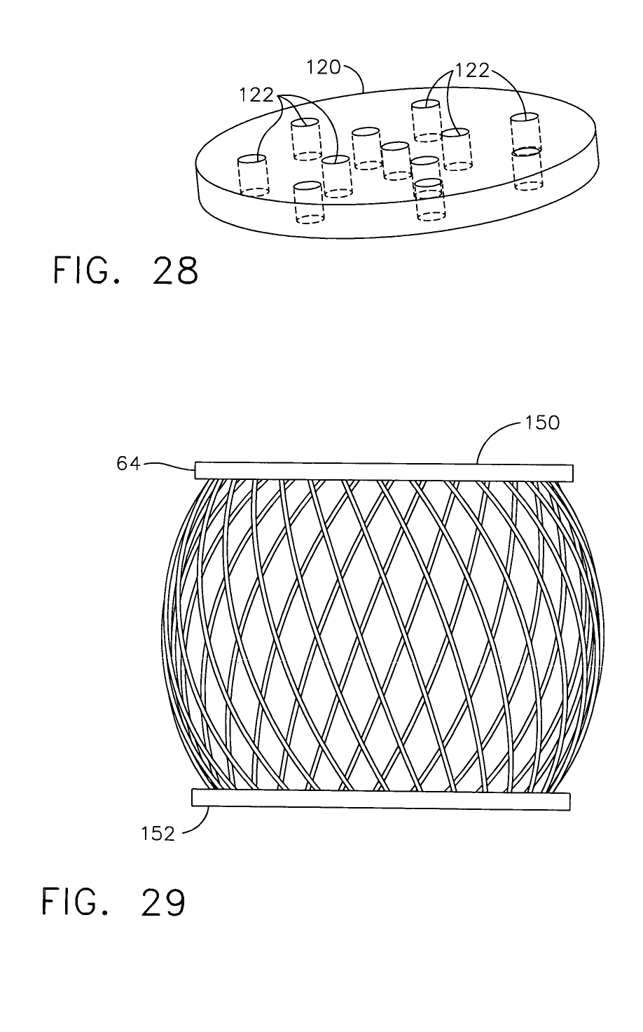

FIG. 28 shows a core 120 which, when molded into shape memory material, will assure mechanical bonding within a thin shell elastomer as shown in FIGS. 16, 17, and 18. Core 120 contains a series of circular channels 122 into which the shape memory material will flow to insure a secure fit within the implant.

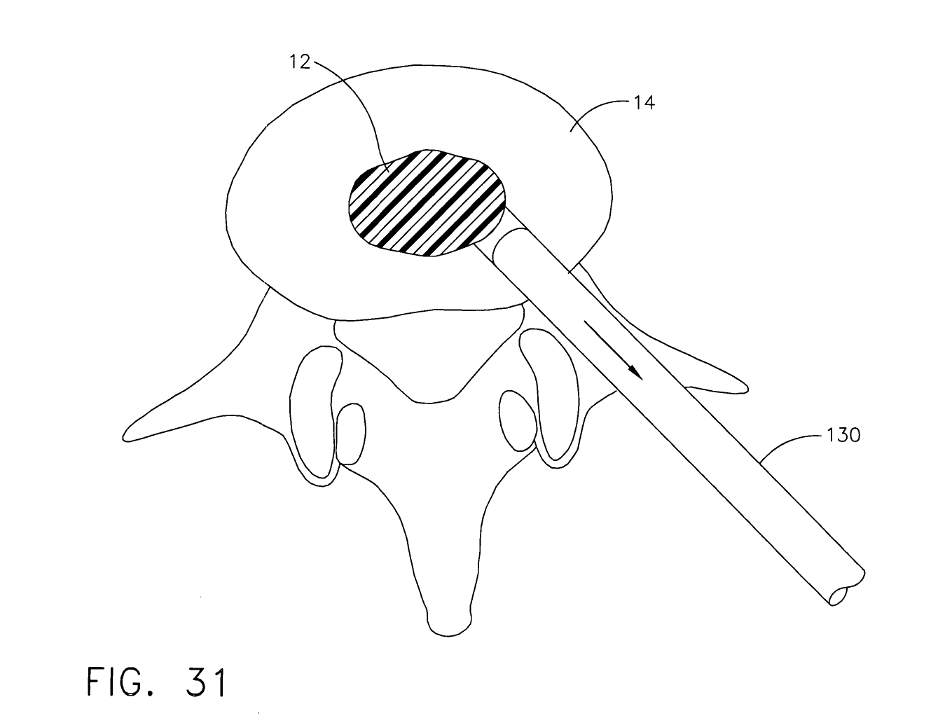

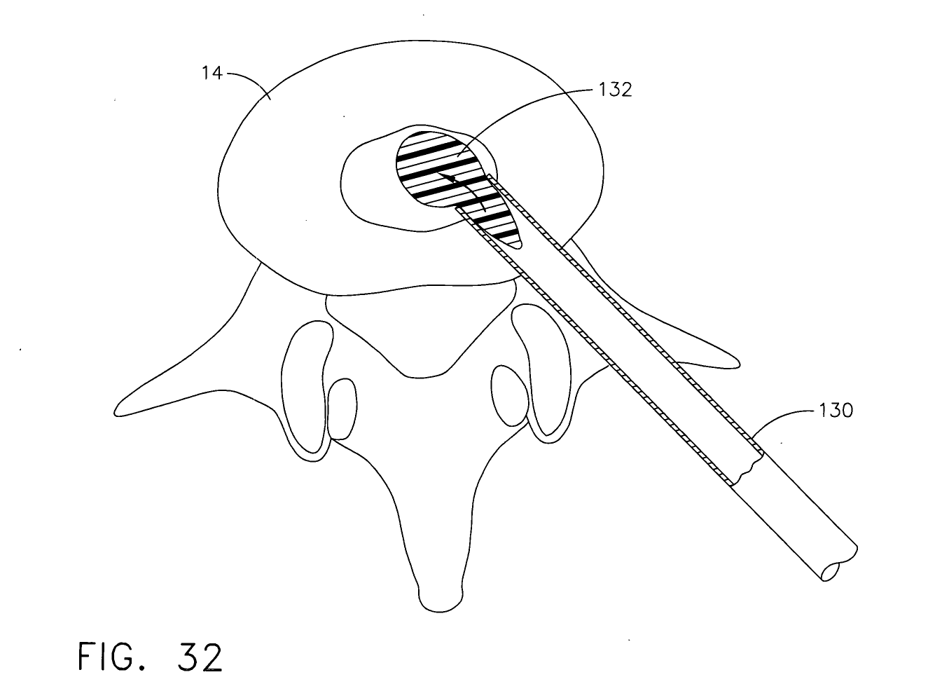

FIGS. 31 and 32 show a delivery device for installing the nucleus implant into its proper location. A cannula device 130 is shown in FIG. 31 removing the cored out section of nucleus 12. The same device 130 can be used to introduce implant 132 into nucleus 12. Device 130 would hold implant 132 in a constrained state and act as a delivery cannula to the cored out cavity 16. Device 130 may also act as the activator of the shape memory material used for implant 132. For example, device 130 could deliver saline at the desired temperature to activate the shape memory material. This technique would allow the material to have a higher temperature, in the active state, such that it is hard and stable when cooled to body temperature. The walls of device 130 can be insulated such that only implant 132 is subjected to the temperature within device 130. Device 130 may have the ability to heat, cool, or transfer other energy to stimulate the material.

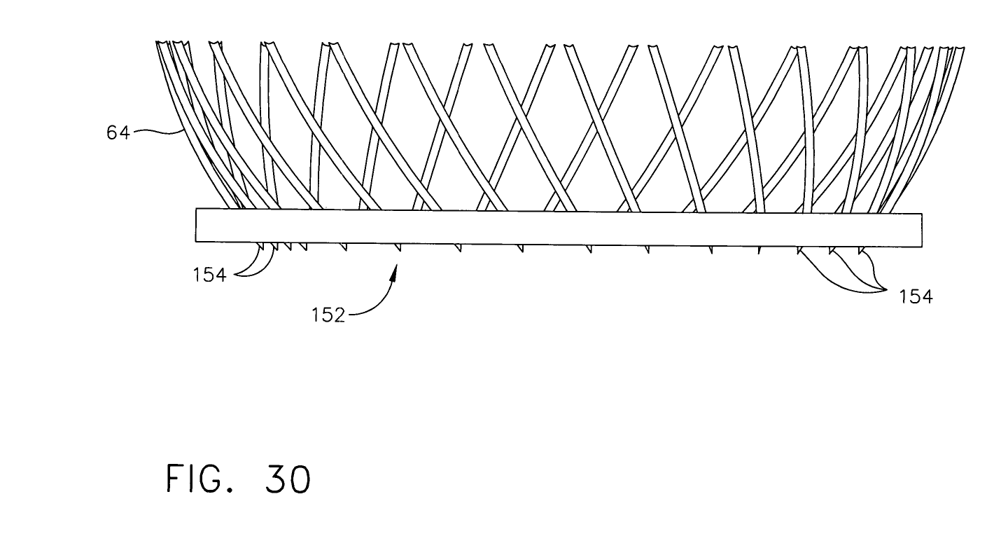

The basket concept of FIGS. 11 and 12 can also be adapted to assist in secure positioning of the implant of the present invention. Referring now to FIGS. 29 and 30, basket 64 contains a polished surface 150 at one end and a rough surface 152 at its other end. Basket 64 is filled with shape memory material when collapsed, and basket 64 is then inserted into hollowed out core 16 of the nucleus of a disc. When the material is activated, basket 64 expands, and rough surface 152 is captured in place on the adjacent vertebra. Surface 152 may also be fitted with a series of prongs 154 to prevent slippage.

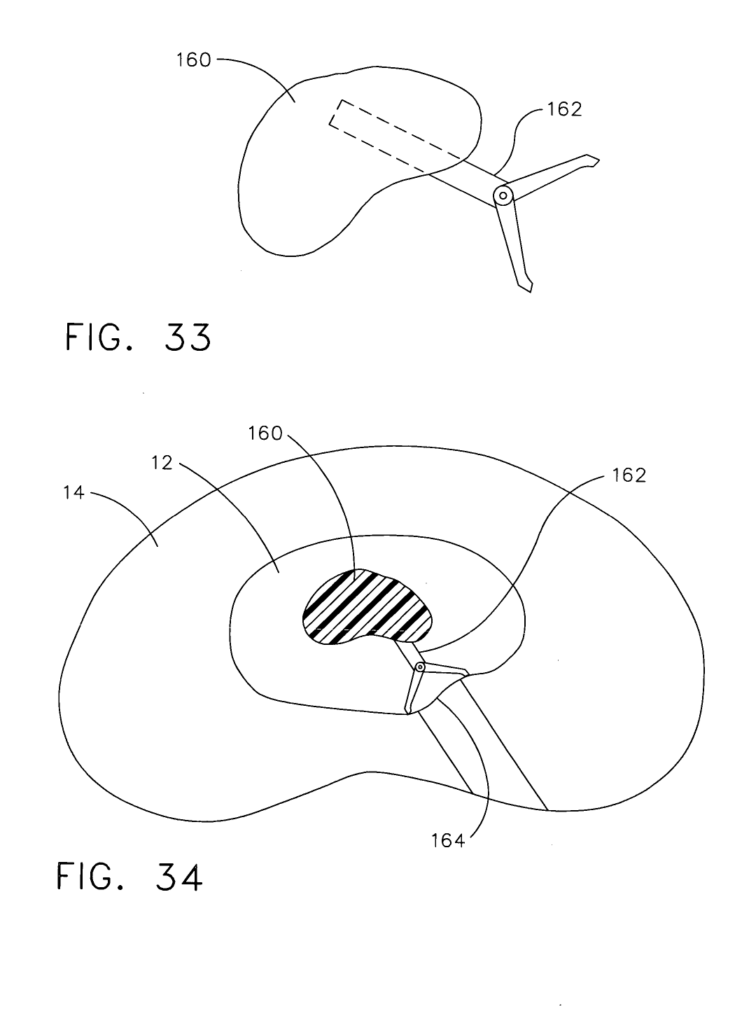

Shape memory material may also be used in combination with other features to improve the stability of a nucleus implant. FIG. 33 shows a combination shape memory polymer 160 into which a metal spring loaded molly 162 has been molded. When the implant is positioned within hollow core section 16 within nucleus 12, molly 162, which may manufactured from stainless steel or nitinol, expands across hernia path 164 in annulus 14, accurately positioning the implant within nucleus 12 and preventing it from leaking out through hernia path 164 in annulus 14.

In summary, some of advantages of the devices and methods of the present invention include: a minimally invasive procedure, meaning less time for incision healing; restores full strength to the spinal disc; allow almost immediate motion and minimal therapy; applications within the spinal disc between the cervical, thoracic, and lumbar vertebrae; and prevents future disc nucleus hernias of the impacted disc.

While the invention has been shown and described in terms of preferred embodiments, it will be understood that this invention is not limited to these particular embodiments and that many changes and modifications may be made without departing from the true spirit and scope of the invention as defined in the appended claims.

1. An intervertebral replacement disc nucleus implant for implantation within the annulus of a spinal disc, comprising:
a shape memory material element having a first shape for easily inserting said element through an opening in the annulus, and a second shape wider than said first shape, which will not pass through said opening in the annulus, wherein said element transforms from said first shape to said second shape by exposure to external energy acting upon said element.

2. The implant of claim 1, wherein said external energy is provided by body heat.

3. The implant of claim 1, wherein said external energy comprises light energy.

4. The implant of claim 1, wherein said external energy comprises thermal energy.

5. The implant of claim 1, wherein said external energy comprises RF energy.

6. The implant of claim 1, wherein said shape memory material comprises a polymer.

7. A method of implanting a disc nucleus replacement device comprised of a shape memory material capable of having a first shape and a second wider shape into a spinal disc of a patient, comprising the steps of:
creating a space within the disc by removing all or a portion of the nucleus tissue;
inserting a cannula into the created space;
transporting the replacement device in the first shape through the cannula into the created space;
and applying external energy to said device to cause the device to transform into said second shape, filling the space created within the disc.

8. The method of claim 7, wherein said external energy is applied by the body of the patient.

9. The method of claim 7, wherein the space creating step is accomplished by ablating the nucleus tissue.

10. The method of claim 9, wherein the nucleus tissue is ablated using RF energy.

11. The method of claim 7, wherein external energy is applied to said device through the cannula.

12. The method of claim 11, wherein said external energy comprises heat energy.

13. The method of claim 11, wherein said external energy comprises light energy.

14. A device for removing tissue from the nucleus of a spinal disc, comprising:
a hollow tube which can be inserted into the nucleus of the disc;
an energy delivering element which can be inserted through said tube into the nucleus;
an RF energy source, coupled to said energy delivering element;
such that when RF energy is applied to said energy delivering element when it is contacting the nucleus tissue, said tissue is ablated, creating an open space within said nucleus.

15. The device of claim 14, wherein said energy delivering element comprises a conductive balloon.

16. The device of claim 14, wherein said energy delivering element comprises a conductive expandable ribbon.

17. A method for removing tissue from the nucleus of a spinal disc, for the purpose of implanting a nucleus implant, comprising the steps of:
inserting a hollow tube into the nucleus of a spinal disc;
inserting an expandable energy delivering element through said tube to contact said nucleus tissue;
applying RF energy to said energy delivering element;
slowly expanding said energy delivering element while RF energy is applied;
and ablating nucleus tissue to create a space within the disc to permit insertion of a nucleus implant.

18. The method of claim 17, wherein said energy delivering element comprises a balloon.

19. The method of claim 17, wherein said energy delivering element comprises an expandable ribbon.

20. The device of claim 1, further comprising securing means, molded into said shape memory material element, such that it is completely enclosed within said element when in said first shape, and extends from said element in said second shape.

21. The device of claim 20, wherein said securing means comprises a prong.

22. The device of claim 21, wherein said prong extends laterally from said shape memory material element.
